(19) United States
(12) Patent Application Publication
(10) Pub. No.: US 2023/0049250 A1
(43) Pub. Date: Feb. 16, 2023

(54) COMPOSITIONS AND METHODS FOR REPAIR OF SPLIT-ENDS IN HAIR

(71) Applicant: Alcora Corporation, Miami, FL (US)

(72) Inventors: Jamie S. ROSS, Homestead, FL (US); Tycho SPEAKER, Santa Cruz, CA (US)

(21) Appl. No.: 16/762,296

(22) PCT Filed: Nov. 13, 2018
(86) PCT No.: PCT/US2018/060643

§ 371 Date: May 7, 2020

Related U.S. Application Data
(60) Provisional application No. 62/584,977, filed on Nov. 13, 2017.

(51) Int. Cl. A61K 8/92; A61K 8/11; A61K 8/73; A61K 8/9717; A61Q 5/00

(52) U.S. Cl. CPC A61K 8/922; A61K 8/11; A61K 8/733; A61K 8/736; A61K 8/73; A61K 8/9717; A61Q 5/002

(57) ABSTRACT

Compositions and methods are disclosed for repairing split ends in hair and/or micro-damage to hair shafts. The compositions comprise a variety of oils, optionally including meadowfoam seed oil (Limnanthes alba seed oil), abyssinian oil (Crambe abyssinica seed oil), and camellia seed oil (Camellia oleifera seed oil). The compositions for repair of split ends and/or micro-damage to hair shafts may be encapsulated in microcapsules. The disclosed compositions and methods can completely repair split ends 3 mm long and under, within one-minute following application.

### CROSS-REFERENCE TO RELATED APPLICATIONS

This application claims the priority benefit of U.S. Provisional Application No. 62/584,977, filed Nov. 13, 2017 and entitled “Compositions and Methods For Repair of Split-Ends in Hair”, which is incorporated herein by reference in its entirety.

### FIELD OF THE INVENTION

The present disclosure relates to the fields of hair care compositions and methods for hair care. More particularly, the present disclosure relates to compositions and methods for repairing damaged hair fibers, such as the split-ends of hair fibers or micro-damage along the shafts of hair fibers.

### BACKGROUND TO THE INVENTION

Damage to hair may result from many factors, including weathering, chemical treatments or mechanical degradation. The splitting of the ends of hair fibers is commonly recognized as a particularly undesirable form of hair damage. During the combing or brushing of the hair, split-ends tend to snarl as the comb or brush approaches the tip ends of the hair and may cause entanglements. Similarly, such entanglements may occur from simply running fingers through hair with split-ends.

Prevention and treatment of split ends generally involves adding lubricity to the hair. Lubrication reduces the friction in the hair, thereby protecting the hair from mechanical damage and reducing entanglements. Yet, addition of lubricity generally does not repair split ends of hair or micro-damage to the shafts of hair.

Once the ends of the hair have split, the options for repair of the split-ends are quite limited. The most common solution is to cut off the split-ends of the hair, but a haircut may not be convenient or desirable.

Thus, there is a need in the art for compositions and methods for repair of split-ends in hair as well as micro-damage to hair shafts.

### SUMMARY OF THE INVENTION

In one aspect, the composition disclosed herein selectively targets and heals split ends and other damaged or stressed areas along the hair shaft, eliminating breakage and frizz, decreasing friction, increasing shine and helping improve color.

In another aspect, the compositions disclosed herein comprise an encapsulated blend of oils effective for repairing split-ends in hair as well as micro-damage to hair shafts.

In some embodiments, the disclosed compositions and methods demonstrate complete repair of split ends 3 mm long and under, within one-minute following application.

In one aspect, a composition for repairing split ends in hair is disclosed comprising a hydrophobic formulation comprising at least 5 components selected from the group consisting of meadowfoam seed oil (Limnanthes alba seed oil), abyssinian oil (Crambe abyssinica seed oil), camellia seed oil (Camellia oleifera seed oil), tomato seed oil (Solanum lycopersicum seed oil), carrot seed oil (Daucus carota sativa seed oil), lemon peel oil (Citrus Limon (Lemon) peel oil), lime oil (Citrus aurantifolia (Lime) oil), bergamot fruit oil (Citrus aurantium bergamia fruit oil), baobab oil (Adansonia digitata oil), buriti fruit oil (Mauritia flexuosa fruit oil), coconut oil (Cocos nucifera oil), Gardenia taitensis flower extract, moringa oil (Moringa oleifera oil), pequi oil (Caryocar brasiliense fruit oil), and sunflower seed oil (Helianthus annuus seed oil), wherein the hydrophobic formulation is optionally encapsulated in a microcapsule wherein the microcapsule comprises a salt of a Lewis acid-Lewis base reactant pair.

In another aspect, a composition for repairing split ends in hair is disclosed, wherein a hydrophobic formulation comprises meadowfoam seed oil (Limnanthes alba seed oil), abyssinian oil (Crambe abyssinica seed oil), camellia seed oil (Camellia oleifera seed oil), tomato seed oil (Solanum lycopersicum seed oil), and carrot seed oil (Daucus carota sativa seed oil).

In another aspect, a composition for repairing split ends in hair is disclosed, wherein a hydrophobic formulation comprises at least 50 weight percent meadowfoam seed oil (Limnanthes alba seed oil).

In another aspect, a composition for repairing split ends in hair is disclosed, wherein a hydrophobic formulation comprises at least 15 weight percent abyssinian oil (Crambe abyssinica seed oil).

In another aspect, a composition for repairing split ends in hair is disclosed, wherein a hydrophobic formulation comprises at least 5 weight percent camellia seed oil (Camellia oleifera seed oil).

In another aspect, a composition for repairing split ends in hair is disclosed, wherein a hydrophobic formulation comprises at least 3 weight percent tomato seed oil (Solanum lycopersicum seed oil).

In another aspect, a composition for repairing split ends in hair is disclosed, wherein a hydrophobic formulation comprises at least 0.5 weight percent carrot seed oil (Daucus carota sativa seed oil).

In another aspect, a composition for repairing split ends in hair is disclosed, wherein a hydrophobic formulation comprises at least 6 components selected from the group consisting of meadowfoam seed oil (Limnanthes alba seed oil), abyssinian oil (Crambe abyssinica seed oil), camellia seed oil (Camellia oleifera seed oil), tomato seed oil (Solanum lycopersicum seed oil), carrot seed oil (Daucus carota sativa seed oil), lemon peel oil (Citrus Limon (Lemon) peel oil), lime oil (Citrus aurantifolia (Lime) oil), bergamot fruit oil (Citrus aurantium bergamia fruit oil), baobab oil (Adansonia digitata oil), buriti fruit oil (Mauritia flexuosa fruit oil), coconut oil (Cocos nucifera oil), Gardenia taitensis flower extract, moringa oil (Moringa oleifera oil), pequi oil (Caryocar brasiliense fruit oil), and sunflower seed oil (Helianthus annuus seed oil).

In another aspect, a composition for repairing split ends in hair is disclosed, wherein a hydrophobic formulation comprises at least 7 components selected from the group consisting of meadowfoam seed oil (Limnanthes alba seed oil), abyssinian oil (Crambe abyssinica seed oil), camellia seed oil (Camellia oleifera seed oil), tomato seed oil (Solanum lycopersicum seed oil), carrot seed oil (Daucus carota sativa seed oil), lemon peel oil (Citrus Limon (Lemon) peel oil), lime oil (Citrus aurantifolia (Lime) oil), bergamot fruit oil (Citrus aurantium bergamia fruit oil), baobab oil (Adansonia digitata oil), buriti fruit oil (Mauritia flexuosa fruit oil), coconut oil (Cocos nucifera oil), Gardenia taitensis flower extract, moringa oil (Moringa oleifera oil), pequi oil (Caryocar brasiliense fruit oil), and sunflower seed oil (Helianthus annuus seed oil).

In another aspect, a composition for repairing split ends in hair is disclosed, wherein a hydrophobic formulation comprises between 0.6 to 2.0 weight percent of Signaline™ S.

In one aspect, a composition for repairing split ends in hair is disclosed comprising a hydrophobic formulation comprising at least 5 components selected from the group consisting of meadowfoam seed oil (Limnanthes alba seed oil), abyssinian oil (Crambe abyssinica seed oil), camellia seed oil (Camellia oleifera seed oil), tomato seed oil (Solanum lycopersicum seed oil), carrot seed oil (Daucus carota sativa seed oil), lemon peel oil (Citrus Limon (Lemon) peel oil), lime oil (Citrus aurantifolia (Lime) oil), bergamot fruit oil (Citrus aurantium bergamia fruit oil), baobab oil (Adansonia digitata oil), buriti fruit oil (Mauritia flexuosa fruit oil), coconut oil (Cocos nucifera oil), Gardenia taitensis flower extract, moringa oil (Moringa oleifera oil), pequi oil (Caryocar brasiliense fruit oil), and sunflower seed oil (Helianthus annuus seed oil), wherein the hydrophobic formulation is optionally encapsulated in a microcapsule wherein the microcapsule comprises a salt of a Lewis acid-Lewis base reactant pair, further wherein the microcapsule comprises algin and chitosan.

In one aspect, a composition for repairing split ends in hair is disclosed comprising a hydrophobic formulation comprising at least 5 components selected from the group consisting of meadowfoam seed oil (Limnanthes alba seed oil), abyssinian oil (Crambe abyssinica seed oil), camellia seed oil (Camellia oleifera seed oil), tomato seed oil (Solanum lycopersicum seed oil), carrot seed oil (Daucus carota sativa seed oil), lemon peel oil (Citrus Limon (Lemon) peel oil), lime oil (Citrus aurantifolia (Lime) oil), bergamot fruit oil (Citrus aurantium bergamia fruit oil), baobab oil (Adansonia digitata oil), buriti fruit oil (Mauritia flexuosa fruit oil), coconut oil (Cocos nucifera oil), Gardenia taitensis flower extract, moringa oil (Moringa oleifera oil), pequi oil (Caryocar brasiliense fruit oil), and sunflower seed oil (Helianthus annuus seed oil), wherein the hydrophobic formulation is optionally encapsulated in a microcapsule wherein the microcapsule comprises a salt of a Lewis acid-Lewis base reactant pair, further wherein the microcapsule comprises algin and chitosan, and further wherein the composition comprises xanthan gum.

In one aspect, a composition for repairing split ends in hair is disclosed comprising a hydrophobic formulation comprising at least 5 components selected from the group consisting of meadowfoam seed oil (Limnanthes alba seed oil), abyssinian oil (Crambe abyssinica seed oil), camellia seed oil (Camellia oleifera seed oil), tomato seed oil (Solanum lycopersicum seed oil), carrot seed oil (Daucus carota sativa seed oil), lemon peel oil (Citrus Limon (Lemon) peel oil), lime oil (Citrus aurantifolia (Lime) oil), bergamot fruit oil (Citrus aurantium bergamia fruit oil), baobab oil (Adansonia digitata oil), buriti fruit oil (Mauritia flexuosa fruit oil), coconut oil (Cocos nucifera oil), Gardenia taitensis flower extract, moringa oil (Moringa oleifera oil), pequi oil (Caryocar brasiliense fruit oil), and sunflower seed oil (Helianthus annuus seed oil), wherein the hydrophobic formulation is optionally encapsulated in a microcapsule wherein the microcapsule comprises a salt of a Lewis acid-Lewis base reactant pair, further wherein the microcapsule comprises algin and chitosan, and further wherein the composition comprises xanthan gum and carrageenan.

In one aspect, a method is disclosed for repairing split ends in hair comprising applying to the hair a composition comprising a hydrophobic formulation comprising at least 5 components selected from the group consisting of meadowfoam seed oil (Limnanthes alba seed oil), abyssinian oil (Crambe abyssinica seed oil), camellia seed oil (Camellia oleifera seed oil), tomato seed oil (Solanum lycopersicum seed oil), carrot seed oil (Daucus carota sativa seed oil), lemon peel oil (Citrus Limon (Lemon) peel oil), lime oil (Citrus aurantifolia (Lime) oil), bergamot fruit oil (Citrus aurantium bergamia fruit oil), baobab oil (Adansonia digitata oil), buriti fruit oil (Mauritia flexuosa fruit oil), coconut oil (Cocos nucifera oil), Gardenia taitensis flower extract, moringa oil (Moringa oleifera oil), pequi oil (Caryocar brasiliense fruit oil), and sunflower seed oil (Helianthus annuus seed oil), wherein the hydrophobic formulation is optionally encapsulated in a microcapsule wherein the microcapsule comprises a salt of a Lewis acid-Lewis base reactant pair.

In one aspect, a method is disclosed wherein the hydrophobic formulation applied to the hair comprises meadowfoam seed oil (Limnanthes alba seed oil), abyssinian oil (Crambe abyssinica seed oil), camellia seed oil (Camellia oleifera seed oil), tomato seed oil (Solanum lycopersicum seed oil), and carrot seed oil (Daucus carota sativa seed oil).

In one aspect, a method is disclosed wherein the hydrophobic formulation applied to the hair comprises at least 6 components selected from the group consisting of meadowfoam seed oil (Limnanthes alba seed oil), abyssinian oil (Crambe abyssinica seed oil), camellia seed oil (Camellia oleifera seed oil), tomato seed oil (Solanum lycopersicum seed oil), carrot seed oil (Daucus carota sativa seed oil), lemon peel oil (Citrus Limon (Lemon) peel oil), lime oil (Citrus aurantifolia (Lime) oil), bergamot fruit oil (Citrus aurantium bergamia fruit oil), baobab oil (Adansonia digitata oil), buriti fruit oil (Mauritia flexuosa fruit oil), coconut oil (Cocos nucifera oil), Gardenia taitensis flower extract, moringa oil (Moringa oleifera oil), pequi oil (Caryocar brasiliense fruit oil), and sunflower seed oil (Helianthus annuus seed oil).

In one aspect, a method is disclosed for repairing split ends in hair comprising applying to the hair a composition comprising a hydrophobic formulation comprising at least 5 components selected from the group consisting of meadowfoam seed oil (Limnanthes alba seed oil), abyssinian oil (Crambe abyssinica seed oil), camellia seed oil (Camellia oleifera seed oil), tomato seed oil (Solanum lycopersicum seed oil), carrot seed oil (Daucus carota sativa seed oil), lemon peel oil (Citrus Limon (Lemon) peel oil), lime oil (Citrus aurantifolia (Lime) oil), bergamot fruit oil (Citrus aurantium bergamia fruit oil), baobab oil (Adansonia digitata oil), buriti fruit oil (Mauritia flexuosa fruit oil), coconut oil (Cocos nucifera oil), Gardenia taitensis flower extract, moringa oil (Moringa oleifera oil), pequi oil (Caryocar brasiliense fruit oil), and sunflower seed oil (Helianthus annuus seed oil), wherein the hydrophobic formulation is optionally encapsulated in a microcapsule wherein the microcapsule comprises a salt of a Lewis acid-Lewis base reactant pair, and further wherein the microcapsule comprises algin and chitosan.

In one aspect, a method is disclosed for repairing split ends in hair comprising applying to the hair a composition comprising a hydrophobic formulation comprising at least 5 components selected from the group consisting of meadowfoam seed oil (Limnanthes alba seed oil), abyssinian oil (Crambe abyssinica seed oil), camellia seed oil (Camellia oleifera seed oil), tomato seed oil (Solanum lycopersicum seed oil), carrot seed oil (Daucus carota sativa seed oil), lemon peel oil (Citrus Limon (Lemon) peel oil), lime oil (Citrus aurantifolia (Lime) oil), bergamot fruit oil (Citrus aurantium bergamia fruit oil), baobab oil (Adansonia digitata oil), buriti fruit oil (Mauritia flexuosa fruit oil), coconut oil (Cocos nucifera oil), Gardenia taitensis flower extract, moringa oil (Moringa oleifera oil), pequi oil (Caryocar brasiliense fruit oil), and sunflower seed oil (Helianthus annuus seed oil), wherein the hydrophobic formulation is optionally encapsulated in a microcapsule wherein the microcapsule comprises a salt of a Lewis acid-Lewis base reactant pair, further wherein the microcapsule comprises algin and chitosan, and further wherein the composition comprises xanthan gum.

In one aspect, a method is disclosed for repairing split ends in hair comprising applying to the hair a composition comprising a hydrophobic formulation comprising at least 5 components selected from the group consisting of meadowfoam seed oil (Limnanthes alba seed oil), abyssinian oil (Crambe abyssinica seed oil), camellia seed oil (Camellia oleifera seed oil), tomato seed oil (Solanum lycopersicum seed oil), carrot seed oil (Daucus carota sativa seed oil), lemon peel oil (Citrus Limon (Lemon) peel oil), lime oil (Citrus aurantifolia (Lime) oil), bergamot fruit oil (Citrus aurantium bergamia fruit oil), baobab oil (Adansonia digitata oil), buriti fruit oil (Mauritia flexuosa fruit oil), coconut oil (Cocos nucifera oil), Gardenia taitensis flower extract, moringa oil (Moringa oleifera oil), pequi oil (Caryocar brasiliense fruit oil), and sunflower seed oil (Helianthus annuus seed oil), wherein the hydrophobic formulation is optionally encapsulated in a microcapsule wherein the microcapsule comprises a salt of a Lewis acid-Lewis base reactant pair, further wherein the composition comprises between 0.6 to 2.0 weight percent of Signaline™ S.

A composition for repairing split ends in hair comprising a hydrophobic formulation comprising at least 5 components selected from the group consisting of meadowfoam seed oil (Limnanthes alba seed oil), abyssinian oil (Crambe abyssinica seed oil), camellia seed oil (Camellia oleifera seed oil), tomato seed oil (Solanum lycopersicum seed oil), carrot seed oil (Daucus carota sativa seed oil), lemon peel oil (Citrus Limon (Lemon) peel oil), lime oil (Citrus aurantifolia (Lime) oil), bergamot fruit oil (Citrus aurantium bergamia fruit oil), baobab oil (Adansonia digitata oil), buriti fruit oil (Mauritia flexuosa fruit oil), coconut oil (Cocos nucifera oil), Gardenia taitensis flower extract, moringa oil (Moringa oleifera oil), pequi oil (Caryocar brasiliense fruit oil), and sunflower seed oil (Helianthus annuus seed oil), wherein the hydrophobic formulation is optionally encapsulated in a microcapsule wherein the microcapsule comprises a salt of a Lewis acid-Lewis base reactant pair and further wherein the composition comprises glycerin.

A composition for repairing split ends in hair comprising a hydrophobic formulation comprising at least 5 components selected from the group consisting of meadowfoam seed oil (Limnanthes alba seed oil), abyssinian oil (Crambe abyssinica seed oil), camellia seed oil (Camellia oleifera seed oil), tomato seed oil (Solanum lycopersicum seed oil), carrot seed oil (Daucus carota sativa seed oil), lemon peel oil (Citrus Limon (Lemon) peel oil), lime oil (Citrus aurantifolia (Lime) oil), bergamot fruit oil (Citrus aurantium bergamia fruit oil), baobab oil (Adansonia digitata oil), buriti fruit oil (Mauritia flexuosa fruit oil), coconut oil (Cocos nucifera oil), Gardenia taitensis flower extract, moringa oil (Moringa oleifera oil), pequi oil (Caryocar brasiliense fruit oil), and sunflower seed oil (Helianthus annuus seed oil), wherein the hydrophobic formulation is optionally encapsulated in a microcapsule wherein the microcapsule comprises a salt of a Lewis acid-Lewis base reactant pair and further wherein the composition comprises glycerin, cetearyl alcohol and behentrimonium chloride.

A composition for repairing split ends in hair comprising a hydrophobic formulation comprising at least 5 components selected from the group consisting of meadowfoam seed oil (Limnanthes alba seed oil), abyssinian oil (Crambe abyssinica seed oil), camellia seed oil (Camellia oleifera seed oil), tomato seed oil (Solanum lycopersicum seed oil), carrot seed oil (Daucus carota sativa seed oil), lemon peel oil (Citrus Limon (Lemon) peel oil), lime oil (Citrus aurantifolia (Lime) oil), bergamot fruit oil (Citrus aurantium bergamia fruit oil), baobab oil (Adansonia digitata oil), buriti fruit oil (Mauritia flexuosa fruit oil), coconut oil (Cocos nucifera oil), Gardenia taitensis flower extract, moringa oil (Moringa oleifera oil), pequi oil (Caryocar brasiliense fruit oil), and sunflower seed oil (Helianthus annuus seed oil), wherein the hydrophobic formulation is optionally encapsulated in a microcapsule wherein the microcapsule comprises a salt of a Lewis acid-Lewis base reactant pair and further wherein the composition comprises glycerin, cetearyl alcohol, behentrimonium chloride, and isoamyl laurate.

A composition for repairing split ends in hair comprising a hydrophobic formulation comprising at least 5 components selected from the group consisting of meadowfoam seed oil (Limnanthes alba seed oil), abyssinian oil (Crambe abyssinica seed oil), camellia seed oil (Camellia oleifera seed oil), tomato seed oil (Solanum lycopersicum seed oil), carrot seed oil (Daucus carota sativa seed oil), lemon peel oil (Citrus Limon (Lemon) peel oil), lime oil (Citrus aurantifolia (Lime) oil), bergamot fruit oil (Citrus aurantium bergamia fruit oil), baobab oil (Adansonia digitata oil), buriti fruit oil (Mauritia flexuosa fruit oil), coconut oil (Cocos nucifera oil), Gardenia taitensis flower extract, moringa oil (Moringa oleifera oil), pequi oil (Caryocar brasiliense fruit oil), and sunflower seed oil (Helianthus annuus seed oil), wherein the hydrophobic formulation is optionally encapsulated in a microcapsule wherein the microcapsule comprises a salt of a Lewis acid-Lewis base reactant pair and further wherein the composition comprises glycerin and between 0.6 to 2.0 weight percent of Signaline™ S.

A haircare composition is disclosed comprising up to 0.5 weight percent, up to 1.0 weight percent, up to 1.5 weight percent, up to 2.0 weight percent, up to 2.5 weight percent, or up to 3.0 weight percent of a composition for repairing split ends in hair comprising a hydrophobic formulation comprising at least 5 components selected from the group consisting of meadowfoam seed oil (Limnanthes alba seed oil), abyssinian oil (Crambe abyssinica seed oil), camellia seed oil (Camellia oleifera seed oil), tomato seed oil (Solanum lycopersicum seed oil), carrot seed oil (Daucus carota sativa seed oil), lemon peel oil (Citrus Limon (Lemon) peel oil), lime oil (Citrus aurantifolia (Lime) oil), bergamot fruit oil (Citrus aurantium bergamia fruit oil), baobab oil (Adansonia digitata oil), buriti fruit oil (Mauritia flexuosa fruit oil), coconut oil (Cocos nucifera oil), Gardenia taitensis flower extract, moringa oil (Moringa oleifera oil), pequi oil (Caryocar brasiliense fruit oil), and sunflower seed oil (Helianthus annuus seed oil), wherein the hydrophobic formulation is optionally encapsulated in a microcapsule wherein the microcapsule comprises a salt of a Lewis acid-Lewis base reactant pair. The haircare composition can be selected from, but is not limited to, the group consisting of a shampoo, a conditioner, a finishing spray, a styling product and combinations thereof.

The following numbered paragraphs [0034]-[0060] contain statements of broad combinations of the inventive technical features herein disclosed:

1. A composition for repairing split ends in hair comprising a hydrophobic formulation comprising at least 5 components selected from the group consisting of meadowfoam seed oil (Limnanthes alba seed oil), abyssinian oil (Crambe abyssinica seed oil), camellia seed oil (Camellia oleifera seed oil), tomato seed oil (Solanum lycopersicum seed oil), carrot seed oil (Daucus carota sativa seed oil), lemon peel oil (Citrus Limon (Lemon) peel oil), lime oil (Citrus aurantifolia (Lime) oil), bergamot fruit oil (Citrus aurantium bergamia fruit oil), baobab oil (Adansonia digitata oil), buriti fruit oil (Mauritia flexuosa fruit oil), coconut oil (Cocos nucifera oil), Gardenia taitensis flower extract, moringa oil (Moringa oleifera oil), pequi oil (Caryocar brasiliense fruit oil), and sunflower seed oil (Helianthus annuus seed oil), wherein the hydrophobic formulation is encapsulated in a microcapsule wherein the microcapsule comprises a salt of a Lewis acid-Lewis base reactant pair.

2. The composition of claim 1, wherein the hydrophobic formulation comprises meadowfoam seed oil (Limnanthes alba seed oil), abyssinian oil (Crambe abyssinica seed oil), camellia seed oil (Camellia oleifera seed oil), tomato seed oil (Solanum lycopersicum seed oil), and carrot seed oil (Daucus carota sativa seed oil).

3. The composition of claim 2, wherein the hydrophobic formulation comprises at least 50 weight percent meadowfoam seed oil (Limnanthes alba seed oil).

4. The composition of claim 3, wherein the hydrophobic formulation comprises at least 15 weight percent abyssinian oil (Crambe abyssinica seed oil).

5. The composition of claim 4, wherein the hydrophobic formulation comprises at least 5 weight percent camellia seed oil (Camellia oleifera seed oil).

6. The composition of claim 5, wherein the hydrophobic formulation comprises at least 3 weight percent tomato seed oil (Solanum lycopersicum seed oil).

7. The composition of claim 6, wherein the hydrophobic formulation comprises at least 0.5 weight percent carrot seed oil (Daucus carota sativa seed oil).

8. The composition of claim 1, wherein the hydrophobic formulation comprises at least 6 components selected from the group consisting of meadowfoam seed oil (Limnanthes alba seed oil), abyssinian oil (Crambe abyssinica seed oil), camellia seed oil (Camellia oleifera seed oil), tomato seed oil (Solanum lycopersicum seed oil), carrot seed oil (Daucus carota sativa seed oil), lemon peel oil (Citrus Limon (Lemon) peel oil), lime oil (Citrus aurantifolia (Lime) oil), bergamot fruit oil (Citrus aurantium bergamia fruit oil), baobab oil (Adansonia digitata oil), buriti fruit oil (Mauritia flexuosa fruit oil), coconut oil (Cocos nucifera oil), Gardenia taitensis flower extract, moringa oil (Moringa oleifera oil), pequi oil (Caryocar brasiliense fruit oil), and sunflower seed oil (Helianthus annuus seed oil).

9. The composition of claim 1, wherein the hydrophobic formulation comprises at least 7 components selected from the group consisting of meadowfoam seed oil (Limnanthes alba seed oil), abyssinian oil (Crambe abyssinica seed oil), camellia seed oil (Camellia oleifera seed oil), tomato seed oil (Solanum lycopersicum seed oil), carrot seed oil (Daucus carota sativa seed oil), lemon peel oil (Citrus Limon (Lemon) peel oil), lime oil (Citrus aurantifolia (Lime) oil), bergamot fruit oil (Citrus aurantium bergamia fruit oil), baobab oil (Adansonia digitata oil), buriti fruit oil (Mauritia flexuosa fruit oil), coconut oil (Cocos nucifera oil), Gardenia taitensis flower extract, moringa oil (Moringa oleifera oil), pequi oil (Caryocar brasiliense fruit oil), and sunflower seed oil (Helianthus annuus seed oil).

10. The composition of claim 1, wherein the microcapsule comprises algin and chitosan.

11. The composition of claim 10, wherein the composition further comprises xanthan gum.

12. The composition of claim 11, wherein the composition further comprises carrageenan.

13. A method of repairing split ends in hair comprising applying to the hair a composition comprising a hydrophobic formulation comprising at least 5 components selected from the group consisting of meadowfoam seed oil (Limnanthes alba seed oil), abyssinian oil (Crambe abyssinica seed oil), camellia seed oil (Camellia oleifera seed oil), tomato seed oil (Solanum lycopersicum seed oil), carrot seed oil (Daucus carota sativa seed oil), lemon peel oil (Citrus Limon (Lemon) peel oil), lime oil (Citrus aurantifolia (Lime) oil), bergamot fruit oil (Citrus aurantium bergamia fruit oil), baobab oil (Adansonia digitata oil), buriti fruit oil (Mauritia flexuosa fruit oil), coconut oil (Cocos nucifera oil), Gardenia taitensis flower extract, moringa oil (Moringa oleifera oil), pequi oil (Caryocar brasiliense fruit oil), and sunflower seed oil (Helianthus annuus seed oil), wherein the hydrophobic formulation is encapsulated in a microcapsule wherein the microcapsule comprises a salt of a Lewis acid-Lewis base reactant pair.

14. The method of claim 13 wherein the hydrophobic formulation comprises meadowfoam seed oil (Limnanthes alba seed oil), abyssinian oil (Crambe abyssinica seed oil), camellia seed oil (Camellia oleifera seed oil), tomato seed oil (Solanum lycopersicum seed oil), and carrot seed oil (Daucus carota sativa seed oil).

15. The method of claim 13, wherein the hydrophobic formulation comprises at least 6 components selected from the group consisting of meadowfoam seed oil (Limnanthes alba seed oil), abyssinian oil (Crambe abyssinica seed oil), camellia seed oil (Camellia oleifera seed oil), tomato seed oil (Solanum lycopersicum seed oil), carrot seed oil (Daucus carota sativa seed oil), lemon peel oil (Citrus Limon (Lemon) peel oil), lime oil (Citrus aurantifolia (Lime) oil), bergamot fruit oil (Citrus aurantium bergamia fruit oil), baobab oil (Adansonia digitata oil), buriti fruit oil (Mauritia flexuosa fruit oil), coconut oil (Cocos nucifera oil), Gardenia taitensis flower extract, moringa oil (Moringa oleifera oil), pequi oil (Caryocar brasiliense fruit oil), and sunflower seed oil (Helianthus annuus seed oil).

16. The method of claim 13, wherein the microcapsule comprises algin and chitosan.

17. The method of claim 16, wherein the composition further comprises xanthan gum.

18. A composition for repairing split ends in hair comprising a hydrophobic formulation comprising at least 5 components selected from the group consisting of meadowfoam seed oil (Limnanthes alba seed oil), abyssinian oil (Crambe abyssinica seed oil), camellia seed oil (Camellia oleifera seed oil), tomato seed oil (Solanum lycopersicum seed oil), carrot seed oil (Daucus carota sativa seed oil), lemon peel oil (Citrus Limon (Lemon) peel oil), lime oil (Citrus aurantifolia (Lime) oil), bergamot fruit oil (Citrus aurantium bergamia fruit oil), baobab oil (Adansonia digitata oil), buriti fruit oil (Mauritia flexuosa fruit oil), coconut oil (Cocos nucifera oil), Gardenia taitensis flower extract, moringa oil (Moringa oleifera oil), pequi oil (Caryocar brasiliense fruit oil), and sunflower seed oil (Helianthus annuus seed oil), wherein the hydrophobic formulation is encapsulated in a microcapsule wherein the microcapsule comprises a salt of a Lewis acid-Lewis base reactant pair and further wherein the composition comprises glycerin.

19. The composition of claim 18, wherein the composition further comprises cetearyl alcohol and behentrimonium chloride.

20. The composition of claim 19, wherein the composition further comprises isoamyl laurate.

21. The composition of claim 1, wherein the composition for repairing split ends further comprises between 0.6 to 2.0 weight percent of Signaline™ S.

22. The method of claim 13, wherein the composition further comprises between 0.6 to 2.0 weight percent of Signaline™ S.

23. The composition of claim 18, wherein the composition for repairing split ends further comprises between 0.6 to 2.0 weight percent of Signaline™ S.

24. A haircare composition comprising up to 1.0 weight percent of the composition for repairing split ends in hair of claim 1.

25. A haircare composition comprising up to 1.5 weight percent of the composition for repairing split ends in hair of claim 1.

26. A haircare composition comprising up to 2.0 weight percent of the composition for repairing split ends in hair of claim 1.

27. The haircare composition of claim 26, wherein the haircare composition is selected from the group consisting of a shampoo, a conditioner, a finishing spray, a styling product and combinations thereof.

### BRIEF DESCRIPTION OF THE DRAWINGS

FIG. 1 illustrates that addition of 1% Signaline™ S to an oil composition as described at Example 1 unexpectedly and significantly improves hyaluronic acid expression compared to either the Rejuvenige™ Oil alone or the Rejuvenige™ Oil with addition of 0.5% Signaline™ S. The hydration of skin, including on the scalp, critically depends on hyaluronic acid protein bound water in the skin. The y-axis of FIG. 1 represents intensity (%) of hyaluronic acid expression detected by staining, wherein untreated skin is 100% and higher staining intensity reflects increased hydration.

FIG. 2 illustrates hyaluronic acid (HA) fluorescent detection in normal human scalp skin after 24 hours of treatment. A 20× objective magnification was used. HA was detected with a fluorescent green color. Panel a of FIG. 2 shows an untreated scalp skin biopsy. Panel b of FIG. 2 shows a scalp skin biopsy treated with Rejuvenige™ oil. Panel c of FIG. 2 shows a scalp skin biopsy treated with Rejuvenige™ S. The pictures are representative of 5 independent experiments.

FIG. 3 illustrates quantification of HA fluorescent detection. With Volocity™ software, statistical analyses, were performed on 5 independent experiments, and were expressed versus “Untreated” (on the left) and versus “Rejuvenige™ oil” (center). The individual data are presented in raw data chapter, Table 4. (Mean±sem; n=25; ***: highly significant with Student's t-test). The application of Rejuvenige™ S for 24 hours was associated with a significant increase of HA expression in normal human scalp skin biopsies, compared to Rejuvenige™ oil and untreated scalp skin.

FIG. 4 illustrates filaggrin immunofluorescent staining in normal human scalp skin after 24 hours of treatment. A 20× objective magnification was used. Filaggrin was detected with a fluorescent green color. Panel a of FIG. 4 shows an untreated scalp skin biopsy. Panel b of FIG. 4 shows a scalp skin biopsy treated with Rejuvenige™ oil. Panel c of FIG. 4 shows a scalp skin biopsy treated with Rejuvenige™ S. The pictures are representative of 5 independent experiments.

FIG. 5 illustrates quantification of filaggrin immunofluorescent staining. With Volocity™ software, statistical analyses, were performed on 5 independent experiments, and were expressed versus “Untreated” (on the left) and versus “Rejuvenige™ oil” (center). The individual data are presented in raw data chapter, Table 5. (Mean±sem; n=25; ***: highly significant with Student's t-test). The application of Rejuvenige™ S for 24 hours was associated with a significant increase of filaggrin expression in normal human scalp skin biopsies, compared to Rejuvenige™ oil and untreated scalp skin.

FIG. 6 illustrates keratin immunofluorescent staining in normal human scalp skin after 24 hours of treatment. A 20× objective magnification was used. Keratin was detected with a fluorescent green color. Panel a of FIG. 6 shows an untreated scalp skin biopsy. Panel b of FIG. 6 shows a scalp skin biopsy treated with Rejuvenige™ oil. Panel c of FIG. 6 shows a scalp skin biopsy treated with Rejuvenige™ S. The pictures are representative of 5 independent experiments.

FIG. 7 illustrates quantification of keratin immunofluorescent staining. With Volocity™ software, statistical analyses, were performed on 5 independent experiments, and were expressed versus “Untreated” (on the left) and versus “Rejuvenige™ oil” (center). The individual data are presented in raw data chapter, Table 6. (Mean±sem; n=25; ***: highly significant with Student's t-test). The application of Rejuvenige™ S for 24 hours was associated with a significant increase of keratin expression in normal human scalp skin biopsies, compared to Rejuvenige™ oil and untreated scalp skin.

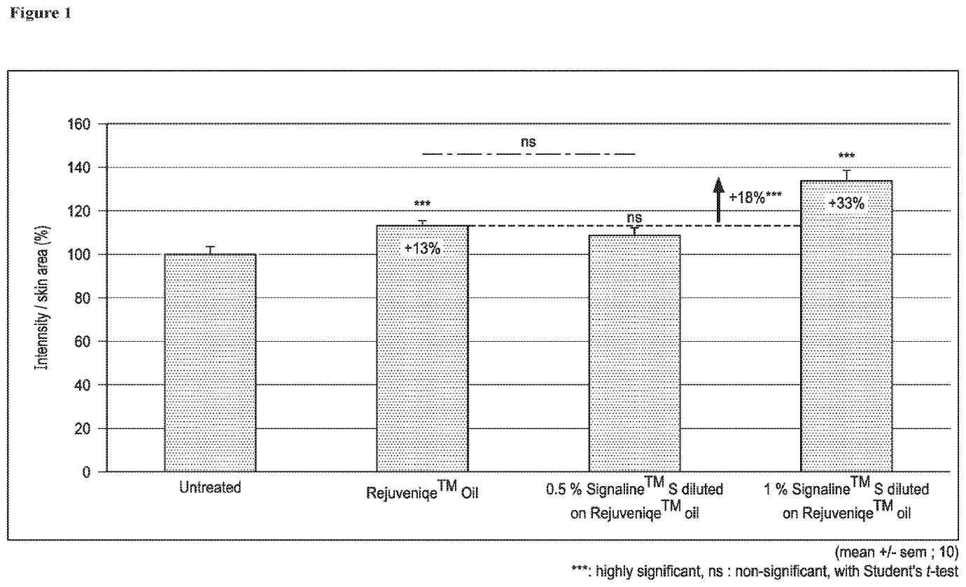

### DETAILED DESCRIPTION

Reference will now be made in detail to representative embodiments of the invention. While the invention will be described in conjunction with the enumerated embodiments, it will be understood that the invention is not intended to be limited to those embodiments. On the contrary, the invention is intended to cover all alternatives, modifications, and equivalents that may be included within the scope of the present invention as defined by the claims.

Unless defined otherwise, technical and scientific terms used herein have the same meaning as commonly understood by one of ordinary skill in the art(s) to which this invention belongs. Although any methods, devices, and materials similar or equivalent to those described herein can be used in the practice or testing of the invention, the preferred methods, devices and materials are now described.

All documents, or portions of documents, cited in this application, including but not limited to patents, patent applications, articles, books, and treatises, are hereby expressly incorporated by reference in their entirety.

As used herein, “a” or “an” may mean one or more than one of an item.

As used herein, “about” may mean up to and including plus or minus five percent, for example, about 100 may mean 95 and up to 105.

As used herein, “encapsulated” means enclosing a material in one or more wall forming reactants.

As used herein, “microcapsule” means a stabilized wall enclosure, of relatively small size which may reasonably be measured with micrometer or nanometer units, which can contain a water-immiscible core phase within an aqueous continuous phase.

As used herein, “Signaline™ S” is an active ingredient mainly composed of: 1,2-Diacylglycerol (1,2-DAG) obtained from olive oil (Olea europaea) and fatty alcohols obtained from jojobawax (Simmondsia chinensis), as described more fully at Patent Cooperation Treaty Publication WO2004/093832.

As used herein, “Rejuvenige™” is a hydrophobic formulation comprising at least 5 components selected from the group consisting of meadowfoam seed oil (Limnanthes alba seed oil), abyssinian oil (Crambe abyssinica seed oil), camellia seed oil (Camellia oleifera seed oil), tomato seed oil (Solanum lycopersicum seed oil), carrot seed oil (Daucus carota sativa seed oil), lemon peel oil (Citrus Limon (Lemon) peel oil), lime oil (Citrus aurantifolia (Lime) oil), bergamot fruit oil (Citrus aurantium bergamia fruit oil), baobab oil (Adansonia digitata oil), buriti fruit oil (Mauritia flexuosa fruit oil), coconut oil (Cocos nucifera oil), Gardenia taitensis flower extract, moringa oil (Moringa oleifera oil), pequi oil (Caryocar brasiliense fruit oil), and sunflower seed oil (Helianthus annuus seed oil).

As used herein, “Rejuvenige™ S” is a hydrophobic formulation comprising Rejuvenige™ and 0.6 to 2.0 weight percent Signaline™ S.

As used herein, “Rejuvabeads™” is an encapsulated form of Rejuvenige™.

In one embodiment, the composition for repairing split-ends of hair and/or micro-damage to hair shafts comprises meadowfoam seed oil (Limnanthes alba seed oil), abyssinian oil (Crambe abyssinica seed oil), camellia seed oil (Camellia oleifera seed oil), tomato seed oil (Solanum lycopersicum seed oil), carrot seed oil (Daucus carota sativa seed oil), lemon peel oil (Citrus Limon (Lemon) peel oil), lime oil (Citrus aurantifolia (Lime) oil), bergamot fruit oil (Citrus aurantium bergamia fruit oil), baobab oil (Adansonia digitata oil), buriti fruit oil (Mauritia flexuosa fruit oil), coconut oil (Cocos nucifera oil), Gardenia taitensis flower extract, moringa oil (Moringa oleifera oil), pequi oil (Caryocar brasiliense fruit oil), and sunflower seed oil (optionally, high in oleic content)(Helianthus annuus seed oil).

Oils listed in the paragraph immediately preceding, were applied to samples of split-end damaged hair (from 20-27 year old Caucasian females) to assess the capacity of the oils to produce repair of the split-end damage. As used herein and throughout the present disclosure, the term “split-end repair” refers specifically to the binding of the two sides of the split hair to restore physical integrity as an intact single fiber as determined by visual inspection using a microscope, and as further determined under extreme magnification in a scanning electron microscope. In no instance did application of any single one of the component oils produce repair of split-end damage. A combination of the component oils was prepared and similarly evaluated. Very surprisingly, split-end damage was repaired upon application of the oil mixture even though none of the individual oils were found to accomplished repair. Without limitation to any particular mechanism of action, one possible explanation for the efficacy of the combination of oils is that the oils individually did not possess the necessary combination of wetting contact angle, plasticization of the hair substrate, penetration into the exposed hair surface, liquid cohesion, and other pertinent physical properties, whereas in contrast, the composition comprising the combined oils struck the correct balance of these properties to achieve visible repair of the split-end damage.

In one aspect, the composition for repairing split-ends of hair and/or micro-damage to hair shafts comprises up to 80 weight percent meadowfoam seed oil (Limnanthes alba seed oil), up to 75 weight percent meadowfoam seed oil, up to 70 weight percent meadowfoam seed oil, up to 65 weight percent meadowfoam seed oil, up to 62 weight percent meadowfoam seed oil, up to 60 weight percent meadowfoam seed oil, up to 55 weight percent meadowfoam seed oil, up to 50 weight percent meadowfoam seed oil, up to 45 weight percent meadowfoam seed oil and/or up to 40 weight percent meadowfoam seed oil.

In another aspect, the composition for repairing split-ends of hair and/or micro-damage to hair shafts comprises up to 30 weight percent abyssinian oil (Crambe abyssinica seed oil), up to 25 weight percent abyssinian oil, up to 22 weight percent abyssinian oil, up to 20 weight percent abyssinian oil, up to 18 weight percent abyssinian oil, up to 15 weight percent abyssinian, and/or up to 10 weight percent abyssinian oil.

In another aspect, the composition for repairing split-ends of hair and/or micro-damage to hair shafts comprises up to 15 weight percent camellia seed oil (Camellia oleifera seed oil), up to 13 weight percent camellia seed oil, up to 11 weight percent camellia seed oil, up to 9 weight percent camellia seed oil, up to 7 weight percent camellia seed oil, up to 5 weight percent camellia seed oil and/or up to 3 weight percent camellia seed oil.

In another aspect, the composition for repairing split-ends of hair and/or micro-damage to hair shafts comprises up to 10 weight percent tomato seed oil (Solanum lycopersicum seed oil), up to 9 weight percent tomato seed oil, up to 7 weight percent tomato seed oil, up to 5 weight percent tomato seed oil, up to 3 weight percent tomato seed oil, and/or up to 1 weight percent tomato seed oil.

In another aspect, the composition for repairing split-ends of hair and/or micro-damage to hair shafts comprises up to 3 weight percent carrot seed oil (Daucus carota sativa seed oil), up to 3 weight percent carrot seed oil, up to 2 weight percent carrot seed oil, up to 1 weight percent carrot seed oil, up to 0.5 weight percent carrot seed oil, and/or up to 0.25 weight percent carrot seed oil.

In another aspect, the composition for repairing split-ends of hair and/or micro-damage to hair shafts comprises up to 4 weight percent lemon peel oil (Citrus Limon (Lemon) peel oil), up to 3 weight percent lemon peel oil, up to 2 weight percent lemon peel oil, up to 1 weight percent lemon peel oil, up to 0.5 weight percent lemon peel oil, and/or up to 0.25 weight percent lemon peel oil.

In another aspect, the composition for repairing split-ends of hair and/or micro-damage to hair shafts comprises up to 4 weight percent lime oil (Citrus aurantifolia (Lime) oil), up to 3 weight percent lime oil, up to 2 weight percent lime oil, up to 1 weight percent lime oil, up to 0.5 weight percent lime oil, and/or up to 0.25 weight percent lime oil.

In another aspect, the composition for repairing split-ends of hair and/or micro-damage to hair shafts comprises up to 4 weight percent bergamot fruit oil (Citrus aurantium bergamia fruit oil), up to 3 weight percent bergamot fruit oil, up to 2 weight percent bergamot fruit oil, up to 1 weight percent bergamot fruit oil, up to 0.5 weight percent bergamot fruit oil, and/or up to 0.25 weight percent bergamot fruit oil.

In another aspect, the composition for repairing split-ends of hair and/or micro-damage to hair shafts comprises up to 1 weight percent baobab oil (Adansonia digitata oil), up to 0.5 weight percent baobab oil, up to 0.25 weight percent baobab oil, up to 0.1 weight percent baobab oil, and/or up to 0.05 weight percent baobab oil.

In another aspect, the composition for repairing split-ends of hair and/or micro-damage to hair shafts comprises up to 1 weight percent buriti fruit oil (Mauritia flexuosa fruit oil), up to 0.5 weight percent buriti fruit oil, up to 0.25 weight percent buriti fruit oil, up to 0.1 weight percent buriti fruit oil, and/or up to 0.05 weight percent buriti fruit oil.

In another aspect, the composition for repairing split-ends of hair and/or micro-damage to hair shafts comprises up to 1 weight percent coconut oil (Cocos nucifera oil), up to 0.5 weight percent coconut oil, up to 0.25 weight percent coconut oil, up to 0.1 weight percent coconut oil, and/or up to 0.05 weight percent coconut oil.

In another aspect, the composition for repairing split-ends of hair and/or micro-damage to hair shafts comprises up to 1 weight percent Gardenia taitensis flower extract, up to 0.5 weight percent Gardenia taitensis flower extract, up to 0.25 weight percent Gardenia taitensis flower extract, up to 0.1 weight percent Gardenia taitensis flower extract, and/or up to 0.05 weight percent Gardenia taitensis flower extract.

In another aspect, the composition for repairing split-ends of hair and/or micro-damage to hair shafts comprises up to 1 weight percent moringa oil (Moringa oleifera oil), up to 0.5 weight percent moringa oil, up to 0.25 weight percent moringa oil, up to 0.1 weight percent moringa oil, and/or up to 0.05 weight percent moringa oil.

In another aspect, the composition for repairing split-ends of hair and/or micro-damage to hair shafts comprises up to 1 weight percent pequi oil (Caryocar brasiliense fruit oil), up to 0.5 weight percent pequi oil, up to 0.25 weight percent pequi oil, up to 0.1 weight percent pequi oil, and/or up to 0.05 weight percent pequi oil.

In another aspect, the composition for repairing split-ends of hair and/or micro-damage to hair shafts comprises up to 1 weight percent sunflower seed oil (optionally, high in oleic content) (Helianthus annuus seed oil), up to 0.5 weight percent sunflower seed oil, up to 0.25 weight percent sunflower seed oil, up to 0.1 weight percent sunflower seed oil, and/or up to 0.05 weight percent sunflower seed oil.

In another aspect, the composition for repairing split-ends of hair and/or micro-damage to hair shafts comprises at least 0.6 weight percent Signaline™ S, at least 0.7 weight percent Signaline™ S, at least 0.8 weight percent Signaline™ S, at least 0.9 weight percent Signaline™ S, at least 1.0 weight percent Signaline™ S, between 0.6 and 3.0 weight percent Signaline™ S, between 0.7 and 2.0 weight percent Signaline™ S, between 0.7 and 1.8 weight percent Signaline™ S, between 0.8 and 1.5 weight percent Signaline™ S, between 0.9 and 1.4 weight percent Signaline™ S, and/or between 0.9 and 1.2 weight percent Signaline™ S, including but not limited to 0.7, 0.8, 0.9, 1.0, 1.1, 1.2, 1.3, 1.4, 1.5, 1.6, 1.7, 1.8, 1.9 and 2.0 weight percent Signaline™ S.

In another aspect, the composition for repairing split-ends of hair and/or micro-damage to hair shafts as described herein may be encapsulated. In one embodiment, the composition is encapsulated in charged polymer microcapsules (CPM). The wall material of the CPM may contain at least one polyfunctional ionic polymer material (i.e., one with multiple charged sites), with charged moieties spaced at intervals along a polymeric chain, which is precipitated by complexation with an oppositely charged partner counterion to form the CPM wall. The wall material may comprise any amino acids, including charged and/or uncharged amino acids.

The encapsulating CPM may be the salt of a Lewis acid-Lewis base reactant pair. A wide variety of combinations of Lewis acid and Lewis base wall materials may be used to produce microcapsules.

Exemplary suitable Lewis acids include, but are not limited to, the following Lewis acids and their anions and water-soluble salts: alginic acid, arabic acid, carboxymethylcellulose, carbomers and related polymers and block copolymers bearing carboxylic acid moieties, hyaluronic acid, gellan gum, pectin, xanthan gum, and water-soluble proteins and/or water-soluble full or partial protein hydrolysates. Suitable exemplary proteins include, but are not limited to, albumin and collagen. Suitable partially hydrolyzed proteins include, but are not limited to, hydrolysates of keratin, milk, rice, soy, silk, and/or wheat proteins. Generally, polymeric materials of molecular weight above 1000 amu presenting at least two carboxylic acid reactive groups, or combinations of these materials, are suitable Lewis acid reactants.

Exemplary suitable Lewis bases include, but are not limited to, the following Lewis bases, anions, and their water-soluble salts: benzalkonium, cetylpyridinium, chitosan, cocodimonium hydroxypropyl hydrolyzed keratin, cocoglucosides hydroxypropyl, hydroxypropyltrimonium hydrolyzed wheat protein, hydroxypropyl oxidized starch PG-trimonium, PEG-3 dioleylamidoethylmonium methosulfate, laurdimoniumhydroxypropyl decylglucosides, polyquaternium-10, polyquaternium-11, polyquaternium-78, polyquaternium-80, polyquaternium-81, polyquaternium-88, polyquaternium-101, quaternium-79 hydrolyzed silk protein, silicone quaternium-17, silicone quaternium-8, starch hydroxypropyltrimonium, steardimonium hydroxyethylcellulose, steardimonium hydroxypropyl panthenyl PEG-7 dimethicone, cocodimonium hydroxyethylcellulose, polyvinylamine and generally, any water-soluble quaternary amine or similar Lewis base capable of forming a salt with the carboxylic acid moieties of the Lewis acids described above, or combinations of any of these materials. Capsule wall materials may further additionally comprise a wide variety of materials that will react with the Lewis acid or Lewis base component. These materials include proteins, partially hydrolyzed proteins, charged amino acids and any of these materials further linked to other compounds that can form salts with the wall materials, some representative examples of which are the quaternized hydrolyzed proteins listed in the exemplary Lewis bases, above. Water-soluble proteins may be used as components of the CPM wall if they can be precipitated by the Lewis base component of the wall. For example, ovalbumin is both readily solubilized and rapidly precipitated by a number of the Lewis bases described.

Microcapsules (CPM) were formed as taught in U.S. Pat. No. 8,039,015, Microencapsulation Product And Process, incorporating in each sample as a core phase one of the individual oils detailed in numbered paragraph [0079] and additionally a sample was prepared with a core phase comprising a combination of those oils. The Lewis acid used for these microcapsules was sodium alginate and the Lewis base used was a commercially available chitosan, derived from a fungal source. The individual encapsulated oils did not reliably produce repair when applied to split end samples. However, surprisingly, the combined oil system provided robust repair that did not separate even upon repeated flexion of the tip of the hair fiber and after washing and drying the fiber. Without limitation to any particular mechanism of action, the combination of oils is very conducive to a strong physical interaction with the keratinaceous material composing the hair, and this interaction may be particularly supportive of hair fiber repair.

The alginate/chitosan wall CPM, further comprising the combined oil system found to repair split ends, was tested for activity on bleach-damaged hair. Control hair, showing intact cuticle layers and no overt damage by scanning electron microscope (SEM) inspection, was treated in a 12% bleach solution for 10 minutes and then rinsed. SEM inspection of the bleached hair showed obvious separation of the cuticle edges from the hair shaft characteristic of bleaching. The bleach-damaged hair was treated by brief (10 second) immersion in the CPM product at a use level corresponding to about 1% of the combined oil system, and then rinsed in distilled water to remove any non-adherent product. Upon re-examination under SEM the treated hair fiber was dramatically changed, with visible smoothing and binding of the previously roughened, uplifted cuticle edges. Without limitation to any particular mechanism of action, the combination of the CPM and the combined oil system appear to result in strong association between the CPM product and the hair fiber resulting not only in repair of split-end macro damage, but also microscopic damage such as that produced by bleaching.

In another aspect, the composition for repairing split-ends of hair and/or micro-damage to hair shafts, encapsulated or unencapsulated, as described herein, may be part of a larger formulation wherein the larger formulation comprises up to 20 weight percent, up to 10 weight percent, up to 5 weight percent, up to 3 weight percent, up to 2 weight percent, and/or up to 1 weight percent of the composition for repair of split ends and/or micro-damage to hair shafts. The larger formulation can be selected from, but is not limited to, the group consisting of a shampoo, a conditioner, a finishing spray, a styling product and combinations thereof. The formulation, comprising the composition for repair of split-ends and/or micro-damage to hair shafts, can deliver the composition for repair of split-ends and/or micro-damage to hair shafts to the ends of hair fibers and/or hair shaft in an efficacious manner.

In one aspect, the larger formulation comprising the composition for repairing split-ends of hair and/or micro-damage to hair shafts comprises up to 95 weight percent water, up to 93 weight percent water, up to 91 weight percent water, up to 89 weight percent water, up to 87 weight percent water, up to 85 weight percent water, up to 83 weight percent water, up to 80 weight percent water, and/or up to 75 weight percent water.

In another aspect, the larger formulation comprising the composition for repairing split-ends of hair and/or micro-damage to hair shafts comprises up to 3 weight percent glycerin, up to 2 weight percent glycerin, up to 1 weight percent glycerin, up to 0.5 weight percent glycerin, and/or up to 0.25 weight percent glycerin.

In another aspect, the larger formulation comprising the composition for repairing split-ends of hair and/or micro-damage to hair shafts comprises up to 9 weight percent cetearyl alcohol, up to 6 weight percent cetearyl alcohol, up to 4 weight percent cetearyl alcohol, up to 2 weight percent cetearyl alcohol, and/or up to 1 weight percent cetearyl alcohol.

In another aspect, the larger formulation comprising the composition for repairing split-ends of hair and/or micro-damage to hair shafts comprises up to 9 weight percent behentrimonium chloride, up to 6 weight percent behentrimonium chloride, up to 4 weight percent behentrimonium chloride, up to 2 weight percent behentrimonium chloride, and/or up to 1 weight percent behentrimonium chloride.

In another aspect, the larger formulation comprising the composition for repairing split-ends of hair and/or micro-damage to hair shafts comprises up to 3 weight percent linseed seed extract (Linum usitatissimum seed extract), up to 2 weight percent linseed seed extract, up to 1 weight percent linseed seed extract, up to 0.5 weight percent linseed seed extract, and/or up to 0.25 weight percent linseed seed extract.

In another aspect, the larger formulation comprising the composition for repairing split-ends of hair and/or micro-damage to hair shafts comprises up to 3 weight percent Salvia hispanica seed extract, up to 2 weight percent Salvia hispanica seed extract, up to 1 weight percent Salvia hispanica seed extract, up to 0.5 weight percent Salvia hispanica seed extract, and/or up to 0.25 weight percent Salvia hispanica seed extract.

In another aspect, the larger formulation comprising the composition for repairing split-ends of hair and/or micro-damage to hair shafts comprises up to 6 weight percent isoamyl laurate, up to 4 weight percent isoamyl laurate, up to 2 weight percent isoamyl laurate, up to 1 weight percent isoamyl laurate, and/or up to 0.5 weight percent isoamyl laurate.

In another aspect, the larger formulation comprising the composition for repairing split-ends of hair and/or micro-damage to hair shafts comprises up to 4 weight percent stearamidopropyl dimethylamine, up to 2 weight percent stearamidopropyl dimethylamine, up to 1 weight percent stearamidopropyl dimethylamine, up to 0.5 weight percent stearamidopropyl dimethylamine, and/or up to 0.25 weight percent stearamidopropyl dimethylamine.

In another aspect, the larger formulation comprising the composition for repairing split-ends of hair and/or micro-damage to hair shafts comprises up to 3 weight percent benzyl alcohol, up to 2 weight percent benzyl alcohol, up to 1 weight percent benzyl alcohol, up to 0.5 weight percent benzyl alcohol, and/or up to 0.25 weight percent benzyl alcohol.

In another aspect, the larger formulation comprising the composition for repairing split-ends of hair and/or micro-damage to hair shafts comprises up to 3 weight percent ethyhexylglycerin, up to 2 weight percent ethyhexylglycerin, up to 1 weight percent ethyhexylglycerin, up to 0.5 weight percent ethyhexylglycerin, and/or up to 0.25 weight percent ethyhexylglycerin.

In another aspect, the larger formulation comprising the composition for repairing split-ends of hair and/or micro-damage to hair shafts comprises up to 3 weight percent tocopherol, up to 2 weight percent tocopherol, up to 1 weight percent tocopherol, up to 0.5 weight percent tocopherol, and/or up to 0.25 weight percent tocopherol.

In another aspect, the larger formulation comprising the composition for repairing split-ends of hair and/or micro-damage to hair shafts comprises up to 10 weight percent fragrance, up to 5 weight percent fragrance, up to 1 weight percent fragrance, up to 0.5 weight percent fragrance, and/or up to 0.25 weight percent fragrance.

In another aspect, the larger formulation comprising the composition for repairing split-ends of hair and/or micro-damage to hair shafts comprises up to 3 weight percent citric acid, up to 2 weight percent citric acid, up to 1 weight percent citric acid, up to 0.5 weight percent citric acid, and/or up to 0.25 weight percent citric acid.

In one embodiment, the composition for repairing split ends and/or micro-damage to hair shafts is soluble in oil.

In one aspect, the composition for repairing split ends and/or micro-damage to hair shafts has a specific gravity of up to 1.2, up to 1.1, up to 1.0, up to 0.9, up to 0.8 and/or up to 0.7. In one embodiment, the composition for repairing split ends and/or micro-damage to hair shafts has a specific gravity of between about 0.900 and 0.910.

In another aspect, the composition for repairing split ends and/or micro-damage to hair shafts has a refractive index of up to 1.9, up to 1.7, up to 1.5, up to 1.3, and/or up to 1.1. In one embodiment, the composition for repairing split ends and/or micro-damage to hair shafts has a refractive index between about 1.465 and 1.475.

In another aspect, the composition for repairing split ends and/or micro-damage to hair shafts has a viscosity of up to 120 cps, up to 110 cps, up to 100 cps, up to 90 cps, up to 80 cps, up to 70 cps, up to 60 cps, and/or up to 50 cps at 72° F. In one embodiment, the composition for repairing split ends and/or micro-damage to hair shafts has a viscosity between 80 and 90 cps at 72° F.

In another aspect, the encapsulated composition for repairing split ends and/or micro-damage to hair shafts has a pH of up to 8, up to 7.5, up to 7.0, up to 6.5, up to 6.0, up to 5.5, up to 5.0, and/or up to 4.5. In one embodiment, the encapsulated composition for repairing split ends and/or micro-damage to hair shafts has a pH between 5.0 and 6.8. In one embodiment, the encapsulated composition for repairing split ends and/or micro-damage to hair shafts has a pH of 5.7.

In another aspect, the encapsulated composition for repairing split ends and/or micro-damage to hair shafts has a specific gravity of up to 1.3, up to 1.2, up to 1.1, up to 1.0, up to 0.9, up to 0.8, up to 0.7 and/or up to 0.6. In one embodiment, the encapsulated composition for repairing split ends and/or micro-damage to hair shafts has a specific gravity between 0.98 and 1.050. In one embodiment, the encapsulated composition for repairing split ends and/or micro-damage to hair shafts has a specific gravity of 0.985.

### Kits

Further embodiments concern kits for use with methods and compositions described herein. Kits comprise the composition for repairing split ends and/or micro-damage to hair shafts. In the kit, the composition for repairing split ends and/or micro-damage to hair shafts can be encapsulated or unencapsulated. The composition for repairing split ends and/or micro-damage to hair shafts may also be part of a larger formulation. The larger formulation may comprise the composition for repairing split ends and/or micro-damage to hair shafts in encapsulated and/or unencapsulated form.

### EXAMPLES

The following examples are provided for illustrative purposes only and are not intended to limit the scope of the invention as defined by the appended claims. All examples described herein were carried out using standard techniques, which are well known and routine to those of skill in the art.

### Example 1

New Oil Blend for Anti-Aging Hair Line with Essential Oil Fragrance Blend

Charge a reactor with all the ingredients from Table 1 and mix until the composition is clear and homogenous.

TABLE 1

| Component | % w/w |
|---|---|
| Limnanthes Alba (Meadowfoam) Seed Oil | 61.85 |
| Crambe Abyssinica Seed Oil | 20.60 |
| Camellia Oleifera Seed Oil | 9.27 |
| Solanum Lycopersicum (Tomato) Seed Oil | 5.15 |
| Daucus Carota Sativa (Carrot) Seed Oil | 1.03 |
| Citrus Limon (Lemon) Peel Oil | 1.00 |
| Citrus aurantifolia (Lime) Oil | 0.50 |
| Citrus Aurantium Bergamia (Bergamot) Fruit Oil | 0.50 |
| Adansonia Digitata Oil | 0.10 |
| Mauritia Flexuosa Fruit Oil | |
| Cocos Nucifera (Coconut) Oil | |
| Gardenia Taitensis Flower Extract | |
| Moringa Oleifera Seed Oil | |
| Caryocar Brasiliense Fruit Oil | |
| Helianthus Annuus (Sunflower) Seed Oil | |
| Total | 100.00 |

### Example 2

Encapsulated Composition for Repairing Split Ends and/or Micro-Damage to Hair Shafts

TABLE 2

| | | Amount | |
|---|---|---|---|
| Ingredients (INCI Name) | Trade Name: | (g) | % w/w |
| Part A: | | | |
| Water | DEI Water | 696.0000 | 87.0000 |
| Glycerin | Glycerin (99%) | 8.0000 | 1.0000 |
| Part B: | | | |
| Cetearyl Alcohol, Behentrimonium Chloride | Incroquat Behenyl TMC-25 | 48.000 | 6.0000 |
| Linum Usitatissimum (Linseed) Seed Extract, | Wavemax | 4.0000 | 0.5000 |
| Salvia Hispanica Seed Extract | | | |
| Isoamyl Laurate | Dermofeel Sensolv | 16.0000 | 2.0000 |
| Stearamidopropyl Dimethylamine | Colonial 18-S | 6.0000 | 0.7500 |
| Part C: | | | |
| Water, Caprylic/Capric Triglyceride, Limnanthes | Rejuvabeads | 12.0000 | 1.5000 |
| Alba (Meadowfoam) Seed Oil, Crambe | | | |
| Abyssinica Seed Oil, Coco-Glucoside, Sorbitan | | | |
| Oleate, Algin, Camellia Oleifera Seed Oil, | | | |
| Solanum Lycopersicum (Tomato) Seed Oil, | | | |
| Daucus Carota Sativa (Carrot) Seed Oil, Citrus | | | |
| Medica Limonum (Lemon) Peel Oil, Citrus | | | |
| Aurantium (Lime) Oil, Citrus Aurantium Bergamia | | | |
| (Bergamot) Fruit Oil, Adansonia Digitata Oil, | | | |
| Mauritia Flexuosa Fruit Oil, Cocos Nucifera | | | |
| (Coconut) Oil, Gardenia Tahitensis Flower | | | |
| Extract, Moringa Oleifera Seed Oil, Caryocar | | | |
| Brasiliense Fruit Oil, Helianthus Annuus | | | |
| (Sunflower) Seed Oil, Guar | | | |
| Hydroxypropyltrimonium Chloride, Chitosan, | | | |
| Xanthan Gum, Carageenan | | | |
| Part D: | | | |
| Benzyl Alcohol, Ethyhexylglycerin, Tocopherol | Euxyl K 900 | 8.0000 | 1.0000 |
| Fragrance | Klabin Pomegranate | 2.0000 | 0.2500 |
| | Sage KF 8532 | | |
| Part E: | | | |
| Citric Acid | Citric Acid (10.0% aq) | q.s. | q.s. |
| Part F: | | | |
| Water | DEI Water | q.s. | q.s. |
| | | 800.0000 | 100.0000 |

Step 1, charge a main reactor with Table 2, Part A ingredients and begin mixing. Begin heating to 70-75° C. Step 2, in a separate reactor melt Table 2, Part B ingredients together to 70-75° C. Step 3, when both Steps 1 and 2 compositions are at temperature, add the composition from Step 2 to the composition of Step 1 and continue mixing. Step 4, mix Step 3 composition until completely melted and uniform. Remove heat and begin cooling. Step 5, cool to below 50° C. and add Table 2, Part C REJUVABEADS. Continue mixing and cooling. Step 6, add Table 2, Part D ingredients mixing well between each. Step 7, adjust pH to 7.5-8.5 with quantum satis (a sufficient quantity) of citric acid solution from Table 2, Part E. Step 8, cool to room temperature and q.s. with water from Table 2, Part F. Mix until uniform. The composition has a pH of 7.5-8.5 and viscosity of 21,000 cPs (6 @ 10).

### Example 3 Microcapsules Comprising Single Oils

250 g of meadowfoam seed oil (Limnanthes alba seed oil) is dispersed into a stirred solution of 670 g of water at 50 C containing 10 g each of sorbitan monooleate and polysorbate 20 to form a creamy emulsion. To the stirring dispersion is added 20 g of a 1% aqueous solution of sodium alginate, followed by 20 g of a 1% aqueous solution of chitosan. The mixture is stirred for 30 minutes and cooled to form the single oil microcapsules described in the present invention. Such microcapsules did not provide robust repair of split ends in testing. Similar microcapsules may be formed by substituting any of the following oils for meadowfoam seed oil: abyssinian oil (Crambe abyssinica seed oil), camellia seed oil (Camellia oleifera seed oil), tomato seed oil (Solanum lycopersicum seed oil), carrot seed oil (Daucus carota sativa seed oil), lemon peel oil (Citrus Limon (Lemon) peel oil), lime oil (Citrus aurantifolia (Lime) oil), bergamot fruit oil (Citrus aurantium bergamia fruit oil), baobab oil (Adansonia digitata oil), buriti fruit oil (Mauritia flexuosa fruit oil), coconut oil (Cocos nucifera oil), Gardenia taitensis flower extract, moringa oil (Moringa oleifera oil), pequi oil (Caryocarbrasiliense fruit oil), or sunflower seed oil (Helianthus annuus seed oil).

### Example 4 Microcapsules Comprising Combined Oils

250 g of an oil mixture consisting of equal parts of meadowfoam seed oil (Limnanthes alba seed oil), abyssinian oil (Crambe abyssinica seed oil), camellia seed oil (Camellia oleifera seed oil), tomato seed oil (Solanum lycopersicum seed oil), carrot seed oil (Daucus carota sativa seed oil), lemon peel oil (Citrus Limon (Lemon) peel oil), lime oil (Citrus aurantifolia (Lime) oil), bergamot fruit oil (Citrus aurantium bergamia fruit oil), baobab oil (Adansonia digitata oil), buriti fruit oil (Mauritia flexuosa fruit oil), coconut oil (Cocos nucifera oil), Gardenia taitensis flower extract, moringa oil (Moringa oleifera oil), pequi oil (Caryocarbrasiliense fruit oil), and sunflower seed oil (Helianthus annuus seed oil), is dispersed into a stirred solution of 670 g of water at 50 C containing 10 g each of sorbitan monooleate and polysorbate 20 to form a creamy emulsion. To the stirring dispersion is added 20 g of a 1% aqueous solution of sodium alginate, followed by 20 g of a 1% aqueous solution of chitosan. The mixture is stirred for 30 minutes and cooled to form the microcapsules of the present invention. This encapsulated form of the combined oils provided a robust repair of split ends in testing, stronger than that obtained with microcapsules of the individual oils used singly.

### Example 5 Microcapsule Components

Charge a reactor with all the ingredients from Table 3 and mix.

TABLE 3

| Component | % w/w |
|---|---|
| Alginate | 0.01% to 2% |
| Chitosan | 0.01% to 2% |
| Oil phase | 0.1% to 30% |
| Sorbitan monooleate | 0.1% to 3% |
| Polysorbate 20 | 0.1% to 3% |

Any combinations of components with the specified microcapsule component ranges of this Example may be used to produce the microcapsules as disclosed herein. Most preferably the materials are combined in the above weight percentages and ratios.

### Example 6 Testing of Combined Oil Compositions Against Encapsulated Combined Oil Compositions

Three independent trials of the combined oil composition were prepared with the following components:

| | Trial | Trial | Trial |
|---|---|---|---|
| Component | 1 | 2 | 3 |
| meadowfoam seed oil (Limnanthes alba seed | 6.66% | 10.00% | 2.00% |
| oil) | | | |
| abyssinian oil (Crambe abyssinica seed oil) | 6.66% | 3.00% | 1.00% |
| camellia seed oil (Camellia oleifera seed oil) | 6.66% | 3.00% | 0.50% |
| tomato seed oil (Solanum lycopersicum seed | 6.66% | 3.00% | 0.50% |
| oil) | | | |
| carrot seed oil (Daucus carota sativa seed oil) | 6.66% | 3.00% | 0.50% |
| lemon peel oil (Citrus Limon (Lemon) peel | 6.66% | 10.00% | 7.50% |
| oil) | | | |
| lime oil (Citrus aurantifolia (Lime) oil) | 6.66% | 10.00% | 7.50% |
| bergamot fruit oil (Citrus aurantium | 6.66% | 3.00% | 0.50% |
| bergamia fruit oil) | | | |
| baobab oil (Adansonia digitata oil) | 6.66% | 3.00% | 0.50% |
| buriti fruit oil (Mauritia flexuosa fruit oil) | 6.66% | 3.00% | 0.50% |
| coconut oil (Cocos nucifera oil) | 6.66% | 25.00% | 70.00% |
| Gardenia taitensis flower extract | 6.66% | 3.00% | 0.50% |
| moringa oil (Moringa oleifera oil) | 6.66% | 3.00% | 0.50% |
| pequi oil (Caryocarbrasiliense fruit oil) | 6.66% | 3.00% | 0.50% |
| sunflower seed oil (Helianthus annuus seed | 6.66% | 15.00% | 7.50% |
| oil) | | | |

For each trial, a portion of the combined oil composition was separated and encapsulated with the encapsulation of Example 5. The combined oil composition was then compared for efficacy with the same combined oil combination encapsulated.

In the case of each trial, the combined oil composition produced repair of two tested split ends such that they did not spontaneously pop open in the charging conditions of the scanning electron beam microscope imaging. None of the samples treated with single oils survived imaging. The combined oil composition alone produced repair of split ends that was durable to rinsing in water in 10 out of 10 tested samples. When subjected to tip-flexion (pressing the tip of the hair fiber along its axis with a finger to produce a sharp curvature of the tip), 9 of these same 10 samples failed, opening the split again.

In the case of each trial, the encapsulated combined oil composition produced repair of two tested split ends that also did not open in the SEM. When 10 split ends were treated with each trial's encapsulated combined oil composition, 100% of the repairs survived tip-flexion stress without opening. In contrast, encapsulated single component oils did not provide repair able to survive SEM and did not survive tip-flexion stressing. Similarly, as above, the hair fiber repair with unencapsulated combined oil composition faired poorly in tip-flexion testing.

There was no difference observed in performance among the three trial combined oil formulations, encapsulated or unencapsulated.

### Example 7 Rejuvenige™ S Evaluation on Normal Human Scalp Skin Biopsies

### 7.1 Materials

7.1.1 Skin biopsies—Normal human skin came from a plastic surgery intervention on the scalp of 5 donors (aged from 42 to 87 year-old). Skin biopsies were obtained with a 6 mm diameter punch (pfm medical). They were cultivated on culture medium containing 50% of DMEM 1 g/L glucose (Lonza™) and 50% of Ham's-F12 (Lonza™) supplemented with 10% of FBS (Lonza™), 2 mM of L-glutamine (Lonza™) and 100 m/mL of Primocin™ (InvivoGen™). Skin biopsies were maintained at 37° C. in a humidified atmosphere containing 5% of CO₂.

7.1.2 Reagents—Biotinylated hyaluronic acid binding protein (bHABP) (Coger™) diluted at 1/400, for 2 hours. Streptavidin-Alexa Fluor™ 488 conjugate (Invitrogen™), diluted at 1/1000, for one hour.

7.1.3 Antibodies—The primary antibodies applied for this study were: Anti-filaggrin (Santacruz™) mouse monoclonal, diluted at 1/100, for an hour and a half. Anti-keratin 15 (Abcam™) rabbit monoclonal, diluted at 1/200, for an hour and a half. The secondary antibodies used were: Alexa Fluor™ 488 donkey anti-mouse (Invitrogen™), diluted at 1/1000, for one hour, Alexa Fluor™ 488 donkey anti-rabbit (Invitrogen™), diluted at 1/1000, for one hour.

### 7.2 HA Fluorescent Detection on Scalp Skin Biopsies

7.2.1 Principle—Hyaluronic acid glycosaminoglycan is detected using the specific, biotinylated, binding protein: bHABP. The recognition of biotin by a streptavidin conjugated to a fluorochrome allows an examination under a fluorescence microscope.

7.2.2 Treatments—Scalp skin biopsies were treated, in duplicate, with the placebo, Rejuvenige™ oil or with Rejuvenige™ S once a day, for 24 hours. 20 μL of solutions were applied on the top of the biopsies.

7.2.3 Biopsies preparation—To allow preservation and section of skin, tissues were fixed for 4 hours in buffered 10% formalin. Samples were transferred to baths with ethanol progressively concentrated to remove water, then followed by two baths of xylene to remove the alcohol, and finally embedded in molten paraffin wax. Embedded skin biopsies were then cut with a microtome (Shandon™) into 4 μm thick sections and placed on Superfrost Plus™ slides (Thermo Scientific™).

7.2.4 Protocol—Sections were deparaffinized and rehydrated with several successive xylene, alcohol and water baths. After a PBS wash, bHABP was applied and slides were incubated for 2 hours, under agitation, at room temperature, in a damp room. After having rinsed slides with PBS, the streptavidin, Alexa Fluor™ 488 conjugate was applied for 1 hour, in the dark, under agitation, at room temperature, in a damp room. Finally, sections were mounted in Fluoromount-G™ (Electron Microscopy Sciences™). Detection was managed and examined using a Zeiss Axiovert™ 200M microscope with a 20× objective. Photos were taken with a Qimaging EXI™ blue camera coupled to Volocity™ acquisition software (Improvision™)

### 7.3 Filaggrin and Keratin 15 Immunofluorescent Staining on Scalp Skin Biopsies

7.3.1 Principle—Immunofluorescence is a technique allowing the visualization of a specific protein in tissue sections by binding a specific primary antibody. A secondary antibody labeled with fluorochrome is used to recognize the primary antibody. Immunofluorescence stained samples are then examined under a fluorescence microscope. A counterstain with DAPI allows to visualize cell nuclei and localize the epidermis.

7.3.2 Treatments—Scalp skin biopsies were treated, in duplicate, with the placebo, Rejuvenige™ oil or with Rejuvenige™ S once a day, for 24 hours. 20 μL of solutions were applied on the top of the biopsies.

7.3.3 Biopsies preparation—To allow preservation and section of skin, tissues were fixed for 4 hours in buffered 10% formalin. Samples were transferred to baths with ethanol progressively concentrated to remove water, then followed by two baths of xylene to remove the alcohol, and finally embedded in molten paraffin wax. Embedded skin biopsies were then cut with a microtome (Shandon™) into 4 μm thick sections and placed on Superfrost Plus™ slides (Thermo Scientific™).

7.3.4 Protocol—Sections were deparaffinized and rehydrated with several successive xylene, alcohol and water baths. Then, an unmasking protocol was performed including 0.25% pepsin (Zymed™, Invitrogen™) digestion for 15 minutes at 37° C. After a PBS wash and saturation of unspecific sites with a solution of 5% BSA (Sigma™) for 30 minutes, the primary antibody was applied and the slides were incubated under agitation, at room temperature, in a damp room. After having rinsed the slides with PBS, the secondary antibody was applied, in the dark, under agitation, at room temperature, in a damp room. Finally, the cell nuclei were stained with 4′,6′-diamidino-2-phenylindole (DAPI, Molecular Probes™) at 0.3 μM for 5 minutes and the sections mounted in Fluoromount-G™ (Electron Microscopy Sciences™). Detection was managed and examined using a Zeiss Axiovert™ 200M microscope with a 20× objective. Photos were taken with a Qimaging EXI™ blue camera coupled to Volocity™ acquisition software (Improvision™)

### 7.4 Image Quantification

Five photos per condition were analyzed with Volocity™ image analysis software (Improvision™), which allows selection of the interesting zone thanks to the fluorescence intensity. The results obtained were the sum of green pixel intensities in the selected zone. Finally, for each photo, the sum obtained was adjusted by considering the area of both examined epidermis and dermis zones for HA, the length of the examined stratum corneum zone for filaggrin and the length of the examined epidermis zone for keratin 15.

### 7.5 Statistical Analysis

Statistical analysis was performed using Student's t test for independent samples with one-tailed direction of rejection for HA and bi-tailed direction of rejection for filaggrin and keratin 15. p≤0.05 were considered as significant, p≤0.01 as very significant and p≤0.005 as highly significant.

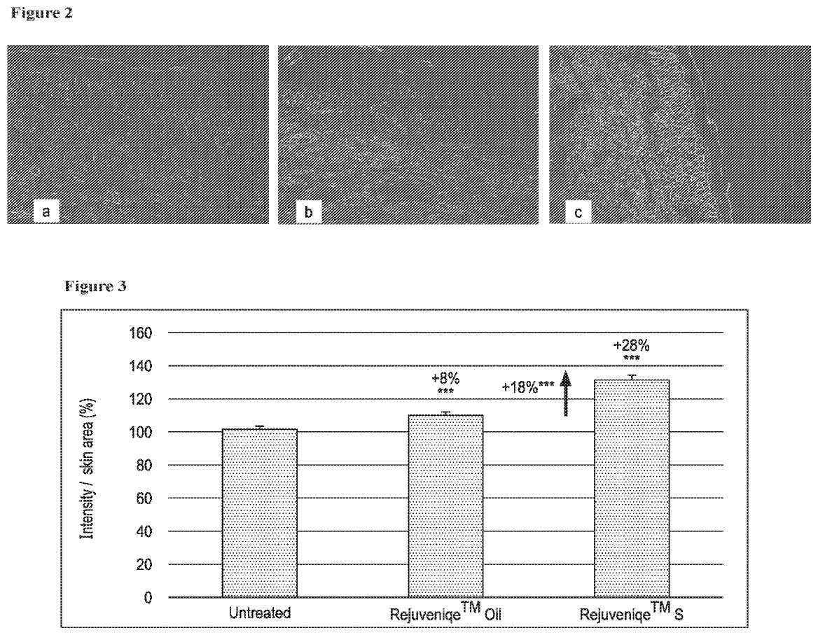

Results of the HA fluorescent detection can be seen at FIG. 2 and FIG. 3. Table 4 provide the quantitative results for the HA fluorescent detection.

TABLE 4
Quantification of HA fluorescent detection

| | | | | | | | | p-value |
|---|---|---|---|---|---|---|---|---|
| | | | | | | | p-value | (Student's |
| | | intensity/ | | intensity/ | | | (Student's t- | t-test) to |
| | nb. | skin area | nb. | cell | | | test) to | Rejuveniqe |
| | value | (%) | value | area (%) | mean | sem | untreated | oil |
| Untreated | 1 | 106.2 | 14 | 98.4 | 100 | 1.9 | | |
| | 2 | 114.1 | 15 | 124.8 | | | | |
| | 3 | 104.7 | 16 | 93.2 | | | | |
| | 4 | 88.9 | 17 | 95.4 | | | | |
| | 5 | 86.1 | 18 | 99.4 | | | | |
| | 6 | 106.6 | 19 | 101.0 | | | | |
| | 7 | 104 | 20 | 110.9 | | | | |
| | 8 | 93.8 | 21 | 82.1 | | | | |
| | 9 | 104.4 | 22 | 107.9 | | | | |
| | 10 | 91.3 | 23 | 101.2 | | | | |
| | 11 | 94.9 | 24 | 101.8 | | | | |
| | 12 | 84.1 | 25 | 107.0 | | | | |
| | 13 | 97.8 | | | | | | |
| Rejuveniqe | 1 | 117 | 14 | 122.7 | 108 | 2.2 | 0.003 | |
| oil | 2 | 119.5 | 15 | 118.6 | | | | |
| | 3 | 111.7 | 16 | 95.8 | | | | |
| | 4 | 103.9 | 17 | 96.3 | | | | |
| | 5 | 102.1 | 18 | 104.5 | | | | |
| | 6 | 121.3 | 19 | 112.1 | | | | |
| | 7 | 123.4 | 20 | 104.8 | | | | |
| | 8 | 117.2 | 21 | 105.9 | | | | |
| | 9 | 115 | 22 | 109.1 | | | | |
| | 10 | 113.3 | 23 | 108.6 | | | | |
| | 11 | 91.6 | 24 | 114.3 | | | | |
| | 12 | 84.5 | 25 | 109.8 | | | | |
| | 13 | 87.6 | | | | | | |
| Rejuveniqe | 1 | 114.2 | 14 | 93.4 | 128 | 3.3 | 0.0000000014 | 0.0000048 |
| S | 2 | 126.9 | 15 | 98.3 | | | | |
| | 3 | 137 | 16 | 136.6 | | | | |
| | 4 | 132 | 17 | 137.8 | | | | |
| | 5 | 134.6 | 18 | 115.6 | | | | |
| | 6 | 162.7 | 19 | 134.8 | | | | |
| | 7 | 160.9 | 20 | 128.2 | | | | |
| | 8 | 143 | 21 | 120.9 | | | | |
| | 9 | 122.1 | 22 | 124.5 | | | | |
| | 10 | 119.9 | 23 | 120.5 | | | | |
| | 11 | 148.4 | 24 | 103.3 | | | | |
| | 12 | 136.2 | 25 | 130.7 | | | | |
| | 13 | 119.6 | | | | | | |

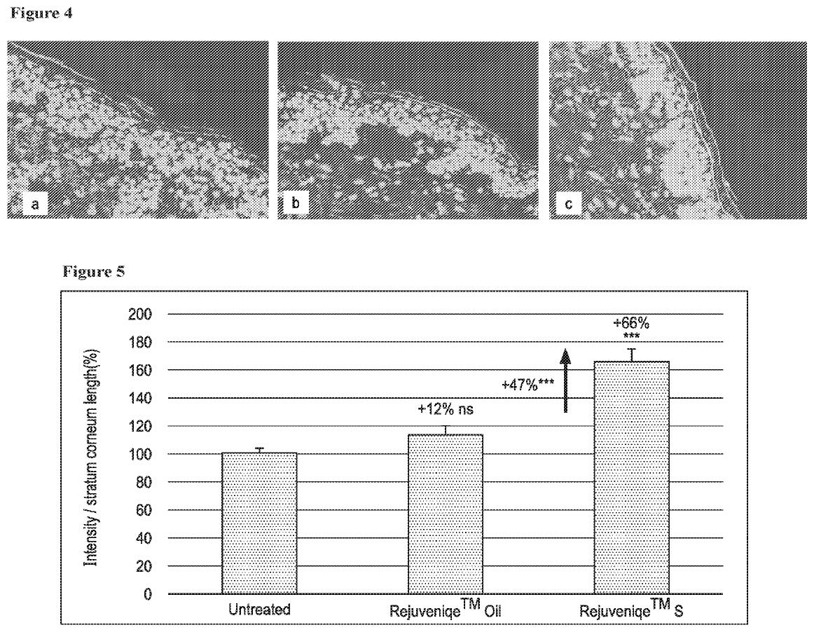

Results of the filaggrin immunofluorescent staining can be seen at FIG. 4 and FIG. 5. Table 5 provides the quantitative results for the filaggrin immunofluorescent staining.

TABLE 5
Quantification of filaggrin immunofluorescent staining

| | | | | | | | | p-value |
|---|---|---|---|---|---|---|---|---|
| | | intensity/ | | intensity/ | | | p-value | (Student's |
| | | stratum | | stratum | | | (Student's | t-test) to |
| | nb. | corneum | nb. | corneum length | | | t-test) to | Rejuveniqe |
| | values | length (%) | values | (%) | Mean | sem | untreated | oil |
| Untreated | 1 | 120.4 | 14 | 84.5 | 100 | 3.5 | | |
| | 2 | 82.9 | 15 | 93.4 | | | | |
| | 3 | 115.2 | 16 | 82.6 | | | | |
| | 4 | 79.4 | 17 | 126.8 | | | | |
| | 5 | 102.1 | 18 | 110.1 | | | | |
| | 6 | 91.6 | 19 | 57.5 | | | | |
| | 7 | 88.1 | 20 | 123 | | | | |
| | 8 | 93.3 | 21 | 111.4 | | | | |
| | 9 | 124.1 | 22 | 87.3 | | | | |
| | 10 | 102.9 | 23 | 104.7 | | | | |
| | 11 | 98.3 | 24 | 110.5 | | | | |
| | 12 | 129.6 | 25 | 86.2 | | | | |
| | 13 | 94.2 | | | | | | |
| Rejuveniqe | 1 | 137.9 | 14 | 163 | 112 | 7.6 | 0.14 | |
| oil | 2 | 117.8 | 15 | 96 | | | | |
| | 3 | 124.6 | 16 | 65.9 | | | | |
| | 4 | 155 | 17 | 51.8 | | | | |
| | 5 | 160 | 18 | 77.8 | | | | |
| | 6 | 92.7 | 19 | 49.6 | | | | |
| | 7 | 100.1 | 20 | 65.7 | | | | |
| | 8 | 87.6 | 21 | 170.8 | | | | |
| | 9 | 84.4 | 22 | 154.8 | | | | |
| | 10 | 81.2 | 23 | 123.5 | | | | |
| | 11 | 117.9 | 24 | 149.1 | | | | |
| | 12 | 84.2 | 25 | 134.7 | | | | |
| | 13 | 165.8 | | | | | | |
| Rejuveniqe | 1 | 151.1 | 14 | 146.9 | 166 | 11.1 | 8.62E−07 | 0.0003 |
| S | 2 | 164.9 | 15 | 127.7 | | | | |
| | 3 | 169.6 | 16 | 93.4 | | | | |
| | 4 | 141.5 | 17 | 70.8 | | | | |
| | 5 | 168.8 | 18 | 84.3 | | | | |
| | 6 | 250.8 | 19 | 105.9 | | | | |
| | 7 | 271 | 20 | 70.9 | | | | |
| | 8 | 172.1 | 21 | 200 | | | | |
| | 9 | 184.9 | 22 | 164.2 | | | | |
| | 10 | 179.5 | 23 | 239.88 | | | | |
| | 11 | 233.3 | 24 | 220.1 | | | | |
| | 12 | 201.5 | 25 | 201.6 | | | | |
| | 13 | 125.1 | | | | | | |

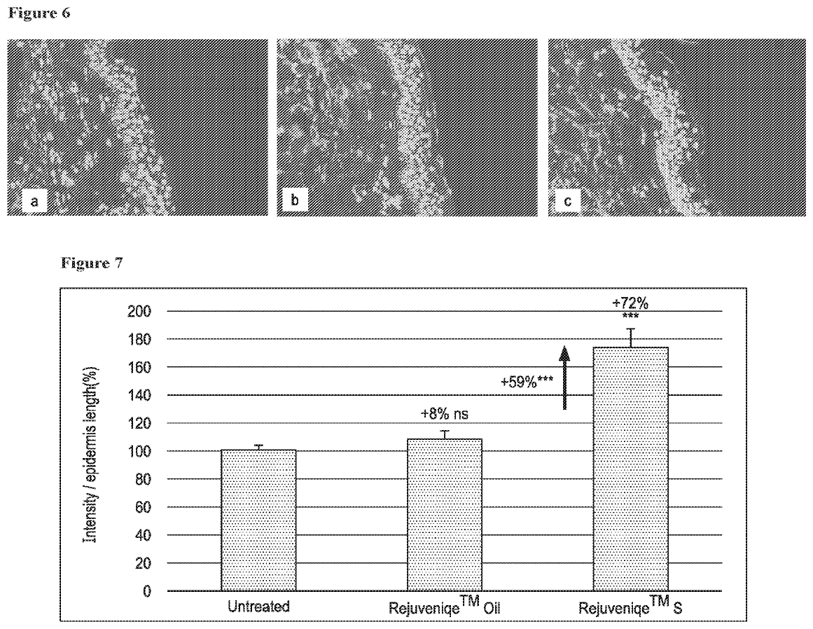

Results of the keratin 15 immunofluorescent staining can be seen at FIG. 6 and FIG. 7. Table 6 provides the quantitative results for the keratin 15 immunofluorescent staining.

TABLE 6
Quantification of keratin 15 immunofluorescent staining

| | | | | | | | | p-value |
|---|---|---|---|---|---|---|---|---|
| | | intensity/ | | intensity/ | | | p-value | (Student's |
| | | epidermis | | epidermis | | | (Student's | t-test) to |
| | nb. | length | nb. | length | | | t-test) to | Rejuveniqe |
| | values | (%) | values | (%) | mean | sem | untreated | oil |
| Untreated | 1 | 94.6 | 14 | 71.8 | 100 | 3.2 | | |
| | 2 | 106.6 | 15 | 116.4 | | | | |
| | 3 | 85.9 | 16 | 129.7 | | | | |
| | 4 | 110.0 | 17 | 99.3 | | | | |
| | 5 | 102.8 | 18 | 85.5 | | | | |
| | 6 | 95.2 | 19 | 92 | | | | |
| | 7 | 137.2 | 20 | 93.4 | | | | |
| | 8 | 82.8 | 21 | 96 | | | | |
| | 9 | 80.6 | 22 | 75.2 | | | | |
| | 10 | 104.2 | 23 | 109.5 | | | | |
| | 11 | 104.4 | 24 | 114.1 | | | | |
| | 12 | 88.7 | 25 | 105.2 | | | | |
| | 13 | 118.7 | | | | | | |
| Rejuveniqe | 1 | 94 | 14 | 55.7 | 108 | 6.9 | 0.32 | |
| oil | 2 | 113.5 | 15 | 47.2 | | | | |
| | 3 | 116.3 | 16 | 71.5 | | | | |
| | 4 | 96.8 | 17 | 92.5 | | | | |
| | 5 | 119.3 | 18 | 160.1 | | | | |
| | 6 | 172.3 | 19 | 109.1 | | | | |
| | 7 | 93.6 | 20 | 127.2 | | | | |
| | 8 | 156.6 | 21 | 89.1 | | | | |
| | 9 | 131.4 | 22 | 132.2 | | | | |
| | 10 | 143 | 23 | 113.7 | | | | |
| | 11 | 55.6 | 24 | 116.6 | | | | |
| | 12 | 79.7 | 25 | 143.9 | | | | |
| | 13 | 59.5 | | | | | | |
| Rejuveniqe | 1 | 332.6 | 14 | 94.6 | 172 | 15.3 | 3.28E−05 | 0.0004 |
| S | 2 | 264.7 | 15 | 58.2 | | | | |
| | 3 | 370.2 | 16 | 15.9 | | | | |
| | 4 | 241 | 17 | 172.4 | | | | |
| | 5 | 277.5 | 18 | 125.9 | | | | |
| | 6 | 180.9 | 19 | 136 | | | | |
| | 7 | 155 | 20 | 125.6 | | | | |
| | 8 | 220.4 | 21 | 130 | | | | |
| | 9 | 167.5 | 22 | 133.8 | | | | |
| | 10 | 156.9 | 23 | 146.7 | | | | |
| | 11 | 126.8 | 24 | 172.3 | | | | |
| | 12 | 85.4 | 25 | 183.4 | | | | |
| | 13 | 75.2 | | | | | | |

Conclusions—Ex vivo tests were conducted on normal human scalp skin biopsies treated for 24 hours with Rejuvenige™ S containing 1% Signaline™ S compared to Rejuvenige™ oil on 5 donors. First, the application of Rejuvenige™ S was associated with an increase in hyaluronic acid expression in scalp biopsies. Hyaluronic acid is involved in skin/scalp hydration maintenance retaining water. Then, the application of Rejuvenige™ S showed an increase in filaggrin expression in scalp biopsies. Filaggrin is known to increase the Natural Moisturizing Factor content in skin and is a key marker of the skin barrier function. At least the application of Rejuvenige™ S demonstrated an increased expression of keratin 15; a biomarker implicated in the epidermal regeneration. These results let us conclude that addition of 1% Signaline™ S to Rejuvenige™ oil boosts scalp health.

1. A composition for repairing split ends in hair comprising a hydrophobic formulation comprising at least 5 components selected from the group consisting of meadowfoam seed oil (Limnanthes alba seed oil), abyssinian oil (Crambe abyssinica seed oil), camellia seed oil (Camellia oleifera seed oil), tomato seed oil (Solanum lycopersicum seed oil), carrot seed oil (Daucus carota sativa seed oil), lemon peel oil (Citrus Limon (Lemon) peel oil), lime oil (Citrus aurantifolia (Lime) oil), bergamot fruit oil (Citrus aurantium bergamia fruit oil), baobab oil (Adansonia digitata oil), buriti fruit oil (Mauritia flexuosa fruit oil), coconut oil (Cocos nucifera oil), Gardenia taitensis flower extract, moringa oil (Moringa oleifera oil), pequi oil (Caryocar brasiliense fruit oil), and sunflower seed oil (Helianthus annuus seed oil), wherein the hydrophobic formulation is encapsulated in a microcapsule wherein the microcapsule comprises a salt of a Lewis acid-Lewis base reactant pair.

2. The composition of claim 1, wherein the hydrophobic formulation comprises meadowfoam seed oil (Limnanthes alba seed oil), abyssinian oil (Crambe abyssinica seed oil), camellia seed oil (Camellia oleifera seed oil), tomato seed oil (Solanum lycopersicum seed oil), and carrot seed oil (Daucus carota sativa seed oil).

3. The composition of claim 2, wherein the hydrophobic formulation comprises
at least 50 weight percent meadowfoam seed oil (Limnanthes alba seed oil);
b) at least 50 weight percent meadowfoam seed oil (Limnanthes alba seed oil) and at least 15 weight percent abyssinian oil (Crambe abyssinica seed oil);
c) at least 50 weight percent meadowfoam seed oil (Limnanthes alba seed oil), at least 15 weight percent abyssinian oil (Crambe abyssinica seed oil), and at least 5 weight percent camellia seed oil (Camellia oleifera seed oil);
d) at least 50 weight percent meadowfoam seed oil (Limnanthes alba seed oil), at least 15 weight percent abyssinian oil (Crambe abyssinica seed oil), at least 5 weight percent camellia seed oil (Camellia oleifera seed oil), and at least 3 weight percent tomato seed oil (Solanum lycopersicum seed oil); or
e) at least 50 weight percent meadowfoam seed oil (Limnanthes alba seed oil), at least 15 weight percent abyssinian oil (Crambe abyssinica seed oil), at least 5 weight percent camellia seed oil (Camellia oleifera seed oil), at least 3 weight percent tomato seed oil (Solanum lycopersicum seed oil), and at least 0.5 weight percent carrot seed oil (Daucus carota sativa seed oil).

4-7. (canceled)

8. The composition of claim 1, wherein the hydrophobic formulation comprises at least 6 components selected from the group consisting of meadowfoam seed oil (Limnanthes alba seed oil), abyssinian oil (Crambe abyssinica seed oil), camellia seed oil (Camellia oleifera seed oil), tomato seed oil (Solanum lycopersicum seed oil), carrot seed oil (Daucus carota sativa seed oil), lemon peel oil (Citrus Limon (Lemon) peel oil), lime oil (Citrus aurantifolia (Lime) oil), bergamot fruit oil (Citrus aurantium bergamia fruit oil), baobab oil (Adansonia digitata oil), buriti fruit oil (Mauritia flexuosa fruit oil), coconut oil (Cocos nucifera oil), Gardenia taitensis flower extract, moringa oil (Moringa oleifera oil), pequi oil (Caryocar brasiliense fruit oil), and sunflower seed oil (Helianthus annuus seed oil).

9. The composition of claim 1, wherein the hydrophobic formulation comprises at least 7 components selected from the group consisting of meadowfoam seed oil (Limnanthes alba seed oil), abyssinian oil (Crambe abyssinica seed oil), camellia seed oil (Camellia oleifera seed oil), tomato seed oil (Solanum lycopersicum seed oil), carrot seed oil (Daucus carota sativa seed oil), lemon peel oil (Citrus Limon (Lemon) peel oil), lime oil (Citrus aurantifolia (Lime) oil), bergamot fruit oil (Citrus aurantium bergamia fruit oil), baobab oil (Adansonia digitata oil), buriti fruit oil (Mauritia flexuosa fruit oil), coconut oil (Cocos nucifera oil), Gardenia taitensis flower extract, moringa oil (Moringa oleifera oil), pequi oil (Caryocar brasiliense fruit oil), and sunflower seed oil (Helianthus annuus seed oil).

10. The composition of claim 1, wherein the microcapsule comprises algin and chitosan.

11. The composition of claim 10, wherein the composition further comprises xanthan gum or carrageenan.

12. (canceled)

13. A method of repairing split ends in hair comprising applying to the hair a composition comprising a hydrophobic formulation comprising at least 5 components selected from the group consisting of meadowfoam seed oil (Limnanthes alba seed oil), abyssinian oil (Crambe abyssinica seed oil), camellia seed oil (Camellia oleifera seed oil), tomato seed oil (Solanum lycopersicum seed oil), carrot seed oil (Daucus carota sativa seed oil), lemon peel oil (Citrus Limon (Lemon) peel oil), lime oil (Citrus aurantifolia (Lime) oil), bergamot fruit oil (Citrus aurantium bergamia fruit oil), baobab oil (Adansonia digitata oil), buriti fruit oil (Mauritia flexuosa fruit oil), coconut oil (Cocos nucifera oil), Gardenia taitensis flower extract, moringa oil (Moringa oleifera oil), pequi oil (Caryocar brasiliense fruit oil), and sunflower seed oil (Helianthus annuus seed oil), wherein the hydrophobic formulation is encapsulated in a microcapsule wherein the microcapsule comprises a salt of a Lewis acid-Lewis base reactant pair.

14. The method of claim 13 wherein the hydrophobic formulation comprises meadowfoam seed oil (Limnanthes alba seed oil), abyssinian oil (Crambe abyssinica seed oil), camellia seed oil (Camellia oleifera seed oil), tomato seed oil (Solanum lycopersicum seed oil), and carrot seed oil (Daucus carota sativa seed oil).

15. The method of claim 13, wherein the hydrophobic formulation comprises at least 6 components selected from the group consisting of meadowfoam seed oil (Limnanthes alba seed oil), abyssinian oil (Crambe abyssinica seed oil), camellia seed oil (Camellia oleifera seed oil), tomato seed oil (Solanum lycopersicum seed oil), carrot seed oil (Daucus carota sativa seed oil), lemon peel oil (Citrus Limon (Lemon) peel oil), lime oil (Citrus aurantifolia (Lime) oil), bergamot fruit oil (Citrus aurantium bergamia fruit oil), baobab oil (Adansonia digitata oil), buriti fruit oil (Mauritia flexuosa fruit oil), coconut oil (Cocos nucifera oil), Gardenia taitensis flower extract, moringa oil (Moringa oleifera oil), pequi oil (Caryocar brasiliense fruit oil), and sunflower seed oil (Helianthus annuus seed oil).

16. The method of claim 13, wherein the microcapsule comprises algin and chitosan.

17. The method of claim 16, wherein the composition further comprises xanthan gum.

18. The composition of claim 1 further comprising glycerin.

19. The composition of claim 18, wherein the composition further comprises cetearyl alcohol and behentrimonium chloride.

20. The composition of claim 19, wherein the composition further comprises isoamyl laurate.

21. The composition of claim 1, wherein the composition for repairing split ends further comprises between 0.6 to 2.0 weight percent of Signaline™ S.

22. The method of claim 13, wherein the composition further comprises between 0.6 to 2.0 weight percent of Signaline™ S.

23. The composition of claim 18, wherein the composition for repairing split ends further comprises between 0.6 to 2.0 weight percent of Signaline™ S.

24. A haircare composition comprising
up to 1.0 weight percent,
up to 1.5 weight percent, or
up to 2.0 weight percent
of the composition for repairing split ends in hair of claim 1.

25-26. (canceled)

27. The haircare composition of claim 24, wherein the haircare composition is selected from the group consisting of a shampoo, a conditioner, a finishing spray, a styling product and combinations thereof.
